(19) United States
(12) Patent Application Publication
(10) Pub. No.: US 2025/0352566 A1
(43) Pub. Date: Nov. 20, 2025

(54) RHAMNOLIPIDS FOR WOUND HEALING

(71) Applicant: Stepan Company, Northbrook, IL (US)

(72) Inventors: Kelly Strout, Lake Zurich, IL (US); Jesse Jefferis, Lincolnshire, IL (US)

(21) Appl. No.: 19/291,876

(22) Filed: Aug. 6, 2025

Related U.S. Application Data
(63) Continuation of application No. PCT/US24/14407, filed on Feb. 5, 2024.
(60) Provisional application No. 63/443,521, filed on Feb. 6, 2023.

(51) Int. Cl. A61K 31/7032; A61K 9/00; A61K 47/10; A61P 17/02

(52) U.S. Cl. CPC A61K 31/7032; A61K 9/0014; A61K 47/10; A61P 17/02

(57) ABSTRACT

The technology presented herein, in general, relates to the use of biosurfactants, such as rhamnolipids, for treating and/or accelerating the healing of wounds. More particularly, the present technology relates to a method for treating wounds, by applying a composition comprising a mixture of particular rhamnolipids to the wound, wherein application of the composition facilitates coordinated gene expression associated with wound healing.

### FIELD OF THE INVENTION

The present technology, in general, relates to the use of biosurfactants, such as rhamnolipids, for treating and/or accelerating the healing of wounds. More particularly, the present technology relates to a method for treating wounds, by applying a composition comprising a mixture of particular rhamnolipids to the wound, wherein application of the composition modifies the gene expression profile associated with wound healing.

### BACKGROUND OF THE INVENTION

The wound-healing process consists of a progression through a number of highly integrated and overlapping phases, including: hemostasis (e.g. vascular constriction, platelet aggregation, degranulation, and fibrin formation (thrombus)); inflammation (e.g., neutrophil infiltration, monocyte infiltration and differentiation to macrophage, and lymphocyte infiltration); proliferation (e.g., re-epithelialization, angiogenesis, collagen synthesis, and ECM formation); and tissue remodeling or resolution (e.g., collagen remodeling, and vascular maturation and regression).

These phases, and their associated biophysiological functions, must occur in the proper sequence, at specific times and durations. Optimal wound healing in adult humans generally involves at least the following the events: (1) rapid hemostasis; (2) appropriate inflammation; (3) mesenchymal cell differentiation, proliferation, and migration to the wound site; (4) suitable angiogenesis; (5) prompt re-epithelialization (re-growth of epithelial tissue over the wound surface); and (6) proper synthesis, cross-linking, and alignment of collagen to provide strength to the healing tissue.

Wounds that exhibit both normal and impaired healing, including both acute and chronic wounds, generally have failed to progress through the normal stages of healing (i.e., enter a state of pathologic inflammation due to a postponed, incomplete, or uncoordinated healing process).

There is therefore an ongoing need for new wound healing compositions, and methods of use, that facilitate both a robust and coordinated healing process, including compositions and methods that facilitate the coordinated activation of genes involved in the normal stages of healing.

There has also been a recent trend to formulate products with ingredients that are based on renewable raw materials. Such ingredients are considered “green” or “natural”, since they are derived from renewable and/or sustainable sources. As a result, they are more environmentally friendly than ingredients derived from fossil fuels or other non-renewable sources. An ingredient having a high Biorenewable Carbon Index (BCI), such as greater than 80, indicates that the ingredient contains carbons that are derived primarily from plant, animal or marine-based sources.

Rhamnolipids are interface-active glycolipids produced by various bacterial species and are an example of a “green” ingredient, since they can be prepared by means of fermentation based on renewable raw materials. It would be desirable to provide compositions that include active ingredients derived from renewable sources, such as rhamnolipids, that can be used to facilitate the treatment and healing of wounds. Providing wound healing/treatment compositions comprising rhamnolipids would satisfy sustainability goals of ensuring sustainable consumption through the use of bio-based antibacterial materials.

Applicants have determined that particular mixtures of rhamnolipid salts can meet the above objectives while also advancing UN Sustainability Goals (“SDG”). The rhamnolipid salt mixtures of the present technology contribute to better health and well-being by delivering equal or better efficacy in the treatment of wounds. The rhamnolipid salt mixtures are advantageously bio-based, renewably sourced actives obtained from a bacterial fermentation process that generates biodegradable waste products that are less impactful on the environment. These benefits further SDG #3 (Good Health and Well-being) and SDG #12 (Responsible Consumption and Production).

### SUMMARY OF THE INVENTION

One aspect of the present technology is directed to compositions and methods for treating a wound. The methods of the present disclosure comprise applying a composition to the wound, wherein the composition comprises at least one rhamnolipid, and wherein application of the composition modifies the expression of at least one gene associated with one or more of the normal stages of healing. In some embodiments, the wound being treated is an incision, a laceration, an abrasion, an avulsion, a puncture, a penetration, or a burn wound. In other embodiments, the composition applied to the wound comprises mono-rhamnolipid, di-rhamnolipid, or a combination of both mono-and di-rhamnolipids. In further embodiments, the expression of at least one gene is increased or decreased in response to the application of the rhamnolipid compositions presented herein.

In another aspect, the present technology is directed to a rhamnolipid composition for use in methods of treating wounds, wherein the composition comprises a mixture of mono-rhamnolipids and di-rhamnolipids having a mono-rhamnolipids: di-rhamnolipids weight ratio of about 40:60 to about 60:40, preferably about 40:60 to about 48:52. In addition, the rhamnolipid composition comprises, based on the total weight of rhamnolipids present in the composition: an amount of C10-C10 mono-rhamnolipid of about 29% to about 40% by weight, preferably 29% to about 37% by weight; an amount of C10-C10 di-rhamnolipid of about 35% to about 50% by weight, preferably about 35% to about 45%; an amount of C8-C10 mono-rhamnolipid of about 2% to about 5% by weight; an amount of C8-C10 di-rhamnolipid of about 2% to about 5% by weight; an amount of C10-C12 mono-rhamnolipid of about 2% to about 6% by weight; and an amount of C10-C12 di-rhamnolipid of about 8% to about 14% by weight. The composition further comprises at least one acceptable carrier, and optionally one or more additives, in an amount to total 100% by weight of the composition.

### BRIEF DESCRIPTION OF THE DRAWINGS

FIG. 1 is a graph showing activation of gene expression, as a percent change relative to 0.9% saline, after a 3-day exposure to Rhamnolipid SEP-RM V2 (with related comparison to 50/50 Mono-rhamnolipid/Di-rhamnolipid mix (0.2% Mono-rhamnolipid (R95Md (Sigma-Aldrich))+0.2% Di-rhamnolipid R95Dd (Sigma-Aldrich) in Saline), and a commercially available comparator rhamnolipid formulation).

FIG. 2 is a graph showing suppression of gene expression, as a percent change relative to 0.9% saline, after a 3-day exposure to Rhamnolipid SEP-RM V2 (with related comparison to 50/50 Mono-rhamnolipid/Di-rhamnolipid mix (0.2% Mono-rhamnolipid (R95Md (Sigma-Aldrich))+0.2% Di-rhamnolipid R95Dd (Sigma-Aldrich) in Saline), and a commercially available comparator rhamnolipid formulation).

FIG. 3 is a graph showing the combined activation and suppression of gene expression, as a percent change relative to 0.9% saline, after a 3-day exposure to Rhamnolipid SEP-RM V2 (with related comparison to 50/50 Mono-rhamnolipid/Di-rhamnolipid mix (0.2% Mono-rhamnolipid (R95Md (Sigma-Aldrich))+0.2% Di-rhamnolipid R95Dd (Sigma-Aldrich) in Saline), and a commercially available comparator rhamnolipid formulation).

FIG. 4 is a graph showing the combined activation and suppression of gene expression, as a percent change relative to 0.9% saline, after a 5-day exposure to Rhamnolipid SEP-RM V2 (with related comparison to 50/50 Mono-rhamnolipid/Di-rhamnolipid mix (0.2% Mono-rhamnolipid (R95Md (Sigma-Aldrich))+0.2% Di-rhamnolipid R95Dd (Sigma-Aldrich) in Saline), and a commercially available comparator rhamnolipid formulation).

FIG. 5 is a graph showing the percent change in gene expression from 3 days to 5 days, relative to 0.9% saline, after exposure to Rhamnolipid SEP-RM V2 (with related comparison to 50/50 Mono-rhamnolipid/Di-rhamnolipid mix (0.2% Mono-rhamnolipid (R95Md (Sigma-Aldrich))+0.2% Di-rhamnolipid R95Dd (Sigma-Aldrich) in Saline), and a commercially available comparator rhamnolipid formulation).

### DETAILED DESCRIPTION OF THE PREFERRED EMBODIMENTS

While the present technology will be described in connection with one or more preferred embodiments, it will be understood by those skilled in the art that the technology is not limited to only those particular embodiments. To the contrary, the presently described technology includes all alternatives, modifications, and equivalents as may be included within the spirit and scope of the appended claims.

Generally, the present disclosure is directed to compositions and methods for treating a wound. The methods disclosed herein comprise applying a composition to a wound, wherein the composition comprises at least one rhamnolipid, and wherein application of the composition to the wound modifies the expression of at least one gene in the area of the wound. Wounds treated herein include, for example, incisions, lacerations, abrasions, avulsions, punctures, penetrations, or burns.

As defined herein, a “rhamnolipid” is a glycolipid that has a lipid portion that includes one or more, typically linear, saturated or unsaturated B-hydroxy-carboxylic acid moieties and a saccharide portion of one or two units of rhamnose.

The saccharide portion and the lipid portion are linked via a B-glycosidic bond between the 1-OH group of a rhamnose moiety of the saccharide portion and the 3-OH group of a B-hydroxy-carboxylic acid of the lipid portion. Thus, the carboxylic acid of one carboxylic acid moiety defines the end of the rhamnolipid. Where more than one rhamnose-moiety is included in a rhamnolipid, each of the rhamnose moieties not linked to the lipid portion is linked to another rhamnose moiety via a 1,4B-glycosidic bond. In embodiments where two or more B-hydroxy-carboxylic acids are present in a rhamnolipid, the B-hydroxy-carboxylic acid moieties are selected independently from each other. B-hydroxy carboxylic acid moieties may in some embodiments be identical. In some embodiments, they are different from each other.

The present technology generally relates to a wound care/treatment composition that comprises a particular mixture of rhamnolipids in their salt form. The rhamnolipids may have the following structure (I):

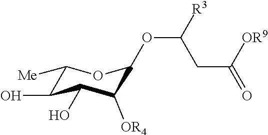

In this formula, R⁹ is a hydrogen atom (H) or an aliphatic group that has a main chain of one to about 46, such as one to about 42, one to about 40, one to about 38, one to about 36, one to about 34, one to about 30, one to about 28, including e.g. 2, 3, 4, 5, 6, 7, 8, 9, 10, 11, 12, 13, 14, 15, 16, 17, 18, 19, 20, 21, 22, 23, 24, 25, 26, 27 or 28 carbon atoms and one to about three, including two, oxygen atoms. In some embodiments, the main chain of the respective aliphatic group carries a terminal carboxylic acid group and/or an internal ester group. As an illustrative example in this regard, R⁹ may be of the formula —CH(R⁵)—CH2—COOR⁶. In these illustrative moieties, R⁵ may be an aliphatic moiety with a main chain that has a length from 1 to about 19, such as from 1 to about 17, from 1 to about 15, from 1 to about 13, about 2 to about 13, about 3 to about 13 or about 4 to about 13, including e.g. 2, 3, 4, 5, 6, 7, 8, 9, 10, 11 or 12 carbon atoms. R⁴ in formula (I) is a hydrogen atom (H), or a rhamnopyranosyl moiety. R⁶ is a hydrogen atom.

The term “aliphatic” means, unless otherwise stated, a straight or branched hydrocarbon chain, which may be saturated or mono-or poly-unsaturated and include heteroatoms. The term “heteroatom” as used herein means an atom of any element other than carbon or hydrogen. Herein, an unsaturated aliphatic group contains one or more double bonds (alkenyl moieties). The branches of the hydrocarbon chain may include linear chains as well as non-aromatic cyclic elements. The hydrocarbon chain, which may, unless otherwise stated, be of any length, and contain any number of branches. Typically, the hydrocarbon (main) chain includes 1 to about 5, to about 10, to about 15 or to about 20 carbon atoms. Examples of alkenyl moieties are straight-chain or branched hydrocarbon moieties that contain one or more double bonds. Alkenyl moieties generally contain about two to about twenty carbon atoms and one or more, for instance two, double bonds, such as about two to about ten carbon atoms, and one double bond. Examples of alkyl groups are methyl, ethyl, propyl, butyl, pentyl, hexyl, heptyl, octyl, nonyl, decyl, the n isomers of these radicals, isopropyl, isobutyl, isopentyl, sec-butyl, tert-butyl, neopentyl, 3,3-dimethylbutyl. Both the main chain as well as the branches may furthermore contain heteroatoms as for instance N, O, S, Se or Si or a carbon atom may be replaced by one of these heteroatoms. An aliphatic moiety may be substituted or unsubstituted with one or more functional groups. Substituents may be any functional group, as for example, but not limited to, amino, amido, carbonyl, carboxyl, hydroxyl, nitro, thio and sulfonyl.

In a more particular embodiment, the rhamnolipid salts in said structure have the structure (II):

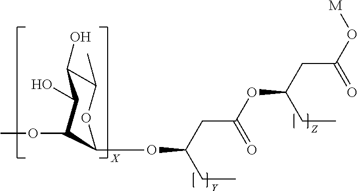

wherein x is 1 or 2, y is 4, 6 or 8, z is 4, 6, or 8, and M is H, or a metal, such as alkali metals Li, Na, or K, alkali earth metals Mg or Ca, or transition metals Mn, Fe, Cu, or Zn. In the cases of the alkali earth and transition metals, multiple rhamnolipid salt moieties may associate with each metal.

The mixture of rhamnolipids preferably comprises mono (where x=1) and di (where x=2) rhamnolipids where y and z are 6 and M is H or Na. The mono-rhamnolipid may be referred to as Rha-C10-C10, with a formula of C₂₆H₄₈O₉. The IUPAC Name is 3-[3-[(2R,3R,4R,5R,6S)-3,4,5-trihydroxy-6-methyloxan-2-yl]oxydecanoyloxy]decanoic acid. The di-rhamnolipid may be referred to as RhaRha-C10-C10, with a formula of C₃₂H₅₈O₁₃. The IUPAC name is 3-[3-[4,5-dihydroxy-6-methyl-3-(3,4,5-trihydroxy-6-methyloxan-2-yl)oxyoxan-2-yl]oxydecanoyloxy]decanoic acid. In general, the mixture of rhamnolipids disclosed herein comprises various types of mono and di rhamnolipids and the mixture specifically encompasses all possible combinations of mono and di rhamnolipids as disclosed herein. Further, unless otherwise stated, an amount of an individual mono or di-rhamnolipid as disclosed herein means that the respective mono or di-rhamnolipid can be present in the mixture of rhamnolipids in the indicated amount, and the mixture of rhamnolipids disclosed herein specifically includes all possible combinations of amounts of mono and di-rhamnolipids as disclosed herein. Generally preferred mixtures of rhamnolipids are SEP-RM rhamnolipid compositions as described below.

Rha-C10-C10 may be present in the mixture in an amount of about 29% to about 40%, alternatively about 30% to about 40%, alternatively about 29% to about 37%, alternatively about 35% to about 37% by weight based on the total weight of rhamnolipids. RhaRha-C10-C10 may be present in the mixture in an amount of about 35% to about 50%, alternatively about 35% to about 45%, alternatively about 36% to about 40%, alternatively about 36% to about 38% by weight based on the total weight of rhamnolipids.

In addition to Rha-C10-C10 and RhaRha-C10-C10, the mixture of rhamnolipids may comprise RhaRha-C10-C12 in an amount of about 8% to about 14%, alternatively about 9% to about 12%, alternatively about 10% to about 12.5% by weight based on the total weight of rhamnolipids, and Rha-C10-C12 in an amount of about 2% to about 6% by weight, alternatively about 2% to about 5%, alternatively about 3.5% to about 5% by weight based on the total weight of rhamnolipids. The mixture of rhamnolipids may also comprise RhaRha-C10-C12:1 in an amount of about 2% to about 5% by weight, alternatively about 3% to about 5% by weight, based on the total weight of rhamnolipids, an amount of RhaRha-C8-C10 in the range of about 2% to about 5% by weight, alternatively about 2% to about 4% by weight, based on the total weight of rhamnolipids, and an amount of Rha-C8-C10 in the range of about 2% to about 5% by weight, alternatively about 2% to about 4% by weight, based on the total weight of rhamnolipids. The mixture of rhamnolipids may also comprise Rha-Rha C12-C12 in an amount of about 0.1% to about 0.5% by weight, alternatively about 0.2% to about 0.4% by weight, alternatively about 0.2% to about 0.3% by weight, alternatively about 0.25% to about 0.3% by weight, based on the total weight of rhamnolipids present in the composition.

The mixture of rhamnolipids may comprise a mixture of mono-rhamnolipids and di-rhamnolipids. The mono-rhamnolipids may be present in an amount of about 40% to about 50%, preferably about 42% to about 48% based on the total weight of rhamnolipids in the mixture. The di-rhamnolipids may be present in an amount of about 50% to about 60% by weight, preferably about 52% to about 58%, based on the total weight of rhamnolipids. The ratio of mono-rhamnolipids: di-rhamnolipids can be from about 40:60 to about 60:40, alternatively about 40:60 to about 50:50, alternatively about 40:60 to about 48:52, preferably about 42:58 to about 48:52.

The terms “active”, “% active”, and “% active weight” refer to the amount of the active ingredient without regard to the amount of water or other solvent that may be present with the ingredient.

As used herein, “effective amount” refers to an amount of an active ingredient or composition that, when administered to a wound, is capable of accelerating or otherwise facilitating the healing process. The actual amount may vary depending on a number of factors, including, but not limited to, the severity of the wound, the age and health status of the subject, and the form of administration.

The mono-rhamnolipid may comprise one or more mono-rhamnolipid-mono-lipidic congeners, including for example: Rha-C8-: 2; Rha-C8; Rha-C10; Rha-C12:2; Rha-C12; Rha-C14:2; or combinations thereof. The mono-rhamnolipid may also comprise one or more mono-rhamnolipid-di-lipidic congeners, including for example: Rha-C8-C8; Rha-C8-C10:1; Rha-C10:1-C8; Rha-C8-C10; Rha-C10-C8; Rha-C10-C10:1; Rha-C10-C10; Rha-C8-C12; Rha-C12-C8; Rha-C10-C12:1; Rha-C12:1-C10; Rha-C10-12; Rha-C12-C10; Rha-C10-C14:1; Rha-C12-C12:1; Rha-C10-C14; Rha-C12-C12; Rha-C12-C14; Rha-C14-C14; Rha-C14-C16; Rha-C16-C16; Rha-C10-C10-CH3; Decenoyl-Rha-C10-C10; or combinations thereof.

The di-rhamnolipid may comprise one or more di-rhamnolipid-mono-lipidic congeners, including for example: Rha-Rha-C8; Rha-Rha-C10; Rha-Rha-C12:1; Rha-Rha-C12; Rha-Rha-C14; or combinations thereof. The di-rhamnolipid may also comprise one or more di-rhamnolipid-di-lipidic congeners, including for example: Rha-Rha-C8-C8; Rha-Rha-C8-C10; Rha-Rha-C10-C8; Rha-Rha-C10-C10:1; Rha-Rha-C10-C10; Rha-Rha-C8-C12:1; Rha-Rha-C12:1-C8; Rha-Rha-C10-C12:1; Rha-Rha-C12:1-C10; Rha-Rha-C10-C12; Rha-Rha-C12-C10; Rha-Rha-C10-C14:1; Rha-Rha-C12-C12:1; Rha-Rha-C12:1-C12; Rha-Rha-C12-C12; Rha-Rha-C12-C14; Rha-Rha-C14-C12; Rha-Rha-C14-C14; Rha-Rha-C14-C16; Rha-Rha-C16-C14; Rha-Rha-C16-C16; Rha-Rha-C14-C14-C14; Rha-Rha-C10-C10-CH3; Decenoyl-Rha-Rha-C10-C10; or combinations thereof.

In one aspect, the present technology provides a wound care composition, and methods of use, wherein the composition comprises a mixture of rhamnolipids in an amount of 0.1% to 99% by weight, based on the total weight of the composition, wherein the mixture of rhamnolipids comprises mono-rhamnolipids and di-rhamnolipids in a weight ratio 40:60 to 60:40 mono-rhamnolipids:di-rhamnolipids, alternatively 40:60 to 50:50 mono-rhamnolipids:di-rhamnolipids, alternatively about 40:60 to about 48:52, alternatively 42:58 to 48:52 mono-rhamnolipids:di-rhamnolipids.

A further aspect of the present technology provides a method for treating a wound comprising administering to the wound an effective amount of a composition comprising a mixture of rhamnolipids, thereby accelerating or otherwise facilitating healing, wherein the mixture of rhamnolipids comprises mono-rhamnolipids and di-rhamnolipids in a weight ratio of 40:60 to 60:40 mono-rhamnolipids:di-rhamnolipids, alternatively 40:60 to 50:50 mono-rhamnolipids:di-rhamnolipids, alternatively 40:60 to 48:52 mono-rhamnolipids:di-rhamnolipids, alternatively 42:58 to 48:52 mono-rhamnolipids:di-rhamnolipids.

In some embodiments, the present technology provides a method for treating a wound, as described above, in which the mixture of rhamnolipids comprises mono-rhamnolipids and di-rhamnolipids in a weight ratio of 42:58 to 48:52, an amount of Rha-C10-C10 mono-rhamnolipid of about 29% to about 40% by weight, and an amount of RhaRha-C10-C10 di-rhamnolipid of about 35% to 50% by weight, based on the total weight of the rhamnolipids in the mixture of rhamnolipids.

The rhamnolipids may be produced from a rhamnolipid-producing microorganism that has the capacity to synthesize/produce rhamnolipids under suitable conditions. Such microorganisms include, but are not limited to, bacteria, particularly bacteria of the phyla Pseudomonadota, Actinobacteria, Fimicutes, and Proteobacteria. The rhamnolipids are naturally derived and therefore have a BCI of 100. In a particular embodiment, the rhamnolipid-producing microorganism for producing the rhamnolipids is Pseudomonas aeruginosa. Methods of culturing the rhamnolipid-producing bacteria and the production of rhamnolipids from fermentation are known in the art from, for example U.S. Pat. Nos. 11,142,782 and 10,144,943, incorporated herein by reference in their entirety. Methods of purifying the rhamnolipids are also known in the art from, for example, U.S. Pat. Nos. 9,884,883 and 10,829,507, incorporated herein by reference in their entirety.

The mixture of rhamnolipid salts can be used alone, as the sole active ingredient in the wound care/treatment composition. When used alone, the mixture of rhamnolipids may be in the range of about 0.01% to about 99% by active weight, based on the total weight of the composition, alternatively about 0.02% to about 25%, alternatively about 0.1% to about 10%, alternatively about 0.2% to about 6% by active weight, based on the total weight of the composition. The mixture of rhamnolipid salts may also be used as a co-active in combination with another active ingredient, such as antibiotics, vitamins (e.g., vitamins E, A, and C), and Hyaluronic Acid. When used in combination, the mixture of rhamnolipids may be in the range of about 0.01% to about 99% by active weight, based on the total weight of the composition, alternatively about 0.02% to about 25%, alternatively about 0.1% to about 10%, alternatively about 0.2% to about 6% by active weight, by active weight based on the total weight of the composition. The combination of the mixture of rhamnolipid salts and another co-active ingredient may help to alleviate the irritation potential of the co-active without reducing or inhibiting its activity. Combining the mixture of rhamnolipid salts with another co-active ingredient may also allow for the reduction of the other co-active ingredient, which can also help to reduce the overall irritation potential.

The wound care/treatment compositions can be formulated into any treatment form commonly used for dermatological/topical applications. For example, the compositions can be in the form of an aqueous solution, suspension, cream, lotion, gel, paste, spray, cream, foam or emollient, or impregnated onto a pads, wipes, bandages and/or dressings.

The wound care/treatment compositions of the present technology also include at least one carrier suitable for wound care/treatment to bring the total percentage of the composition to 100%. As will be appreciated by at least those skilled in the art, a variety of carriers, vehicles, diluents, and the like are suitable for use in the practice of the present technology. Thus, it will also be appreciated that the terms “carrier”, “vehicle”, and “diluent” are to be considered non-exhaustive and interchangeable with respect to the present technology and in describing the various formulations, applications, uses, and compositions thereof.

Water is a suitable carrier, and can be de-ionized water, hard water, soft water, distilled water, tap water or combinations thereof. Water can be used alone as the carrier, or in combination with other carriers suitable for personal care, such as for example, alcohols such as ethanol, isopropanol, or benzyl alcohol; glycols such as propylene glycol, or polyethylene glycol. Other carriers can include, but are not limited to solvents, emulsifiers, or solubilizers.

When the treatment form is a cream, gel, or paste, the wound care/treatment compositions can include, but are not limited to, vegetable gums, starches, celluloses, waxes, silicone, silica, or clays, as carrier ingredients. When the treatment form is a spray, the composition may include a propellant.

In addition to the rhamnolipid active and carrier, the wound care/treatment compositions of the present technology can include optional ingredients as known in the art. Such other components or additives can include, but are not limited to, surfactants, pH adjustment agents, skin conditioners, antioxidants, preservatives, fragrances, pigments, dyes, and other excipients (e.g., anesthetics such as Benzocaine, and other antibiotics).

The wound care/treatment compositions of the present technology can have pH values in the range of about 4.0 to about 8.5, alternatively, about 5.0 to about 8.0, ideally 5.5 to 7.0.

The wound care/treatment compositions of the present technology may be used by applying the composition to the wound of a subject in an amount effective to treat, and/or otherwise facilitate wound healing. “Applying” can refer to any commonly used method of application, such as, but not limited to, spreading a cream or gel containing the wound care/treatment composition on the surface of the wound and allowing the cream or gel to remain on the wound; spraying a liquid containing the wound care/treatment composition on the surface of the wound and, if desired, surrounding tissue; wiping the wound with a wipe impregnated with the wound care/treatment composition and allowing the composition to remain on the wound and, if desired, surrounding tissue; applying a pad impregnated with the wound care/treatment composition and allowing the pad to remain on the surface of the wound and, if desired, surrounding tissue; or an aqueous or nonaqueous liquid wash intended to treat the surface of the wound and, if desired, the surrounding tissue.

Dosage forms and treatment regimens using the wound care/treatment compositions of the present technology can vary with the type and intensity of the wound. In one or more embodiments, methods of treatment in accordance with the present technology may use a one, two, three, four, or more daily dosage regime. The daily dosage regimen can continue for 1-6 days, alternatively one, two, three, four, five, six, or more weeks according to the condition and response of the patient.

Methods of the present disclosure comprise applying a rhamnolipid containing wound care/treatment composition to a wound, wherein application of the composition modifies the expression of at least one gene associated with one or more of the normal stages of healing, including: hemostasis (e.g. vascular constriction, platelet aggregation, degranulation, and fibrin formation (thrombus)); inflammation (e.g., neutrophil infiltration, monocyte infiltration and differentiation to macrophage, and lymphocyte infiltration); proliferation (e.g., re-epithelialization, angiogenesis, collagen synthesis, and ECM formation); and tissue remodeling or resolution (e.g., collagen remodeling, and vascular maturation and regression).

In one aspect of the present disclosure, the application of a rhamnolipid composition contemplated herein results in the activation or repression of gene expression for at least one of the following genes: actin alpha 2 (ACTA2); ADAM metallopeptidase domain 17 (ADAM17); bone morphogenetic protein 6 (BMP6); CD14 molecule (CD14); claudin 1 (CLDN1); heparin binding EGF like growth factor (HBEGF); hepatocyte growth factor (HGF); hypoxia inducible factor 1 subunit alpha (HIF1A); intercellular adhesion molecule 1 (ICAM1); integrin subunit beta 1 (ITGB1); occludin (OCLN); plakophilin 1 (PKP1); peroxisome proliferator activated receptor delta (PPARD); transforming growth factor beta 1 (TGFB1); toll like receptor 3 (TLR3), or combinations thereof.

### ITEMS OF THE INVENTION

The invention further relates to the following items:
1. A rhamnolipid composition for use in methods of treating wounds, wherein the composition comprises a mixture of mono-rhamnolipids and di-rhamnolipids having a mono-rhamnolipids: di-rhamnolipids weight ratio of about 40:60 to about 60:40. The rhamnolipid composition also comprises, based on the total weight of rhamnolipids present in the composition, an amount of C10-C10 mono-rhamnolipid of about 29% to about 40% by weight, an amount of C10-C10 di-rhamnolipid of about 35% to about 50% by weight, an amount of C8-C10 mono-rhamnolipid of about 2% to about 5% by weight; an amount of C8-C10 di-rhamnolipid of about 2% to about 5% by weight; an amount of C10-C12 mono-rhamnolipid of about 2% to about 6% by weight; and an amount of C10-C12 di-rhamnolipid of about 8% to about 14% by weight. The composition also further comprises at least one acceptable carrier, and optionally one or more additives, in an amount to total 100% by weight of the composition.
2. The composition of the preceding item, wherein the mixture of rhamnolipids comprises an amount of total mono rhamnolipid of about 40% to about 50% by weight, preferably about 40% to about 48% by weight, such as about 42% to about 48% by weight, or about 43% to about 47% by weight, or about 43% to about 45% by weight, based on the total weight of rhamnolipids present in the composition.
3. The composition of any one of the preceding items, wherein the mixture of rhamnolipids comprises an amount of total di-rhamnolipid of about 50% to about 60% by weight, preferably about 52% to about 60% by weight, such as about 52% to about 58% by weight, or about 53% to about 57% by weight, or about 55% to about 57% by weight, based on the total weight of rhamnolipids present in the composition.
4. The composition of any one of the preceding items, wherein the mixture of rhamnolipids comprises an amount of the mono-rhamnolipid C8-C10 congener in the range of about 2% to about 4% by weight, based on the total weight of rhamnolipids present in the composition.
5. The composition of any one of the preceding items, wherein the mixture of rhamnolipids comprises an amount of the mono-rhamnolipid C10-C10 congener in the range of about 29% to about 37% by weight, preferably about 32% to about 37% by weight, such as about 34% to about 37% by weight, or about 35% to about 37% by weight, based on the total weight of rhamnolipids present in the composition.
6. The composition of any one of the preceding items, wherein the mixture of rhamnolipids comprises an amount of the mono-rhamnolipid C10-C12 congener in the range of about 2% to about 5% by weight, preferably about 3.5% to about 5% by weight, based on the total weight of rhamnolipids present in the composition.
7. The composition of any one of the preceding items, wherein the mixture of rhamnolipids comprises an amount of the di-rhamnolipid C8-C10 congener in the range of about 2% to about 4% by weight, based on the total weight of rhamnolipids present in the composition.
8. The composition of any one of the preceding items, wherein the mixture of rhamnolipids comprises an amount of the di-rhamnolipid C10-C10 congener in the range of about 35% to about 45% by weight, preferably about 36% to about 40% by weight, such as about 36% to about 38% by weight, based on the total weight of rhamnolipids present in the composition.
9. The composition of any one of the preceding items, wherein the mixture of rhamnolipids comprises an amount of the di-rhamnolipid C10-C12.1 congener in the range of about 2% to about 5% by weight, preferably about 3% to about 5% by weight, such as about 3% to about 4% by weight, based on the total weight of rhamnolipids present in the composition.
10 The composition of any one of the preceding items, wherein the mixture of rhamnolipids comprises an amount of the di-rhamnolipid C10-C12 congener in the range of about 9% to about 12% by weight, preferably in the range of about 10% to about 12% by weight, or about 10% to about 12.5% by weight, based on the total weight of rhamnolipids present in the composition.
11. The composition of any one of the preceding items, wherein the mixture of rhamnolipids comprises an amount of the di-rhamnolipid C12-C12 congener in the range of about 0.1% to about 0.5% by weight, preferably about 0.2% to about 0.4% by weight, such as about 0.2% to about 0.3% by weight, or about 0.25% to about 0.3% by weight, based on the total weight of rhamnolipids present in the composition.
12. The composition of any one of the preceding items, wherein the mixture of rhamnolipids is a SEP-RM Rhamnolipid composition.
13. The composition of any one of the preceding items, wherein the mixture of rhamnolipids is a SEP-RM Rhamnolipid composition obtained by a solvent extraction process including a bleaching step after acidulation and before the solvent extraction.

### EXAMPLES

The presently described technology and its advantages will be better understood by reference to the following examples. These examples are provided to describe specific embodiments of the present technology. By providing these examples, the inventors do not limit the scope and spirit of the present technology.

### Experimental Procedures ***

### Test Materials

### Rhamnolipid Compositions

Rhamnolipids are produced, as understood in the art, through the fermentation of rhamnolipid producing bacteria such as Pseudomonas aeruginosa. The rhamnolipids may be produced from a rhamnolipid-producing microorganism that has the capacity to synthesize/produce rhamnolipids under suitable conditions. Such microorganisms include, but are not limited to, bacteria, particularly bacteria of the phyla Pseudomonadota, Actinobacteria, Fimicutes, and Proteobacteria. The rhamnolipids are naturally derived and therefore have a BCI of 100. In a particular embodiment, the rhamnolipid-producing microorganism for producing the rhamnolipids is Pseudomonas aeruginosa. Methods of culturing the rhamnolipid-producing bacteria and the production of rhamnolipids from fermentation are known in the art from, for example U.S. Pat. Nos. 11,142,782 and 10,144,943, incorporated herein by reference in their entirety. Methods of purifying the rhamnolipids are also known in the art from, for example, U.S. Pat. Nos. 9,884,883 and 10,829,507, incorporated herein by reference in their entirety.

Fermentation whole broth then undergoes multiple purification steps. The specific combination of steps depends on the rhamnolipid actives and purity requirements of the target application. Certain rhamnolipid mixtures used herein are purified by a solvent extraction process. As used herein, the acronym SEP-RM refers to Solvent Extraction Purified Rhamnolipid Mixture (or composition), in accordance with the current specification.

For example, the SEP-RM Rhamnolipid compositions used herein are prepared in accordance with at least the following processing steps: (1) fermentation of appropriate Rhamnolipid producing bacterium; (2) biomass separation; (3) sterilization; (4) clarification (e.g. filtration); (5) acidulation; (6) bleaching; (7) washing; (8) solvent extraction; (9) neutralization and dilution; and (10) final polishing step(s). As discussed below, the solvent extraction process/step is intended to yield a higher purity rhamnolipid mixture/composition for personal care applications. The additional solvent extraction steps provide a rhamnolipid composition having a lighter color and milder odor profile that are preferred for the personal care market.

At the end of fermentation, the whole broth typically contains rhamnolipids along with biomass and other by-products of fermentation. To separate the biomass solids, the broth can be centrifuged. The resulting centrifuged broth is then subjected to sterilization (e.g., high temperature sterilization), after which, the centrifuged sterilized broth is clarified by filtration to remove suspended solids.

Further purification is achieved by treating the clarified broth with acid, which converts the rhamnolipid to a water-insoluble form that settles to the bottom and separates from the bulk aqueous phase. This dense acidulated rhamnolipid is then isolated (and referred to as Acidulated, Concentrated Clarified Broth (ACCB). ACCB is then treated with bleaching agent, then water washed to remove residual bleaching agent and other water-soluble impurities. At this point, the decolorized washed ACCB is concentrated (e.g., ≥about 45% actives, or between about 35% to about 55% actives) and purer (e.g., ≥about 75% purity, or between about 65% to about 85% purity).

Solvent Extraction Process—To achieve better purity, color, and odor, the decolorized Acidulated, Concentrated Clarified Broth (ACCB) undergoes a solvent extraction process. This is generally performed by dissolving the decolorized, washed ACCB in organic solvent, preferably ethyl acetate. The rag layer that typically forms is separated from the bulk solution. Activated carbon is then added to the rhamnolipid solution in ethyl acetate for further decolorization and deodorization. The slurry is filtered and the resulting solution is stripped under vacuum to remove ethyl acetate. The highly concentrated ACCB obtained as residue is then neutralized and diluted (e.g., from between about 20% to about 30% actives, preferably about 25% actives) to give crude SEP-RM. As a final polishing step, crude SEP-RM is then washed with ethyl acetate to extract the antifoam and yield the final product as optically clear SEP-RM at about 25% active and about 85% purity.

### Mono-Rhamnolipid: Di-Rhamnolipid Ratio and Congener Distribution

The mono-rhamnolipid: di-rhamnolipid ratio, and congener distribution for SEP-RM rhamnolipid compositions of the present disclosure can be determined using HPLC (High Performance Liquid Chromatography). As understood by the person of ordinary skill in the art, HPLC will separate rhamnolipid congeners by chain length and number of rhamnose moieties (with the more polar congeners eluting first). Fractions are collected and analyzed by mass spec to identify each peak. The congener distribution is reported as area percent (with actives being calculated relative to known standards).

The HPLC congener distribution for SEP-RM rhamnolipid compositions of the present technology may comprise mono-rhamnolipid C8-C10 congener in an amount of about 2% to about 5% by weight, di-rhamnolipid C8-C10 congener in an amount of about 2% to about 5% by weight, mono-rhamnolipid C10-C10 congener in an amount of about 29% to about 40% by weight, preferably about 35% to about 40% by weight, di-rhamnolipid C10-C10 congener in an amount of about 35% to about 50 weight, preferably about 35% to about 45% by weight, mono-rhamnolipid C10-C12 congener in an amount of about 2% to about 6% by weight, di-rhamnolipid C10-C12 congener in an amount of about 8% to about 14% by weight, with total mono-rhamnolipid congeners in an amount of about 40% to about 48% by weight, and total di-rhamnolipid congeners in an amount of about 52% to about 60% by weight, based on the total weight of the congeners in the composition.

The HPLC congener distribution for an exemplary SEP-RM rhamnolipid composition of the present disclosure, is presented below:

SEP-RM (Solvent Extraction Purified - Rhamnolipid Mixture)

| Congener | HPLC Relative Area % |
|---|---|
| Di (C8C10) | 3.21 |
| Mono (C8C10) | 3.29 |
| Di (C10C10) | 37.10 |
| Mono (C10C10) | 36.71 |
| Di(C10:C12:1) | 3.82 |
| Di (C10C12) | 11.18 |
| Mono (C10C12) | 4.41 |
| Di(C12:C12) | 0.28 |
| Total Mono-Rhamnoliid wt % | 44.41 |
| Total Di-rhamnolipid wt % | 55.59 |

Optional Bleaching Step—SEP-RM V1 versus SEP-RM V2

The SEP-RM rhamnolipid composition provided herein can be processed either with, or without a bleaching step (occurring after acidulation, and before the solvent extraction step(s)). As used herein, SEP-RM VI refers to a SEP-RM rhamnolipid composition prepared and processed without a bleaching step. As used herein, SEP-RM V2 refers to a SEP-RM rhamnolipid composition prepared and processed with a bleaching step (occurring after acidulation, and before the solvent extraction step(s)). For the SEP-RM rhamnolipid compositions of the present disclosure, the mono-rhamnolipid: di-rhamnolipid ratio, and the associated congener distribution, are unaffected by whether a bleaching step is included (V2), or not included (V1), in the purification process.

The following groups were included in the study (N=4 per time point): Negative Control (0.9% Saline); 0.2% Stepan Rhamnolipid SEP-RM V1 (i.e., SEP-RM rhamnolipid composition described above, without added bleaching step)) in Saline; 0.2% Stepan Rhamnolipid SEP-RM V2 (i.e., SEP-RM rhamnolipid composition described above, with added bleaching step (occurring after acidulation, and before the solvent extraction)) in Saline; 0.2% Mono-rhamnolipid R95Md (Sigma-Aldrich) in Saline; 0.2% Di-rhamnolipid R95Dd (Sigma-Aldrich) in Saline; artificially formulated 50/50 Mono-rhamnolipid/Di-rhamnolipid mix (0.2% Mono-rhamnolipid (R95Md (Sigma-Aldrich))+0.2% Di-rhamnolipid R95Dd (Sigma-Aldrich) in Saline); and a commercially available comparator rhamnolipid formulation. The test materials were stored at room temperature and protected from light until use. The working stock of each test material was prepared to the desired concentration fresh before application using 0.9% saline (Moltox, lot #56205) or media (Mattek lot #100322GSH).

Tissue Equilibrium—EFT-400 tissues (MatTek lot #34294, kits FF, EE, and Z-HCF) were equilibrated overnight at 37° C. with 5% CO₂ and ˜95% relative humidity. The following day, equilibration medium was removed from each well and replaced with 2.5 ml fresh maintenance medium (MatTek EFT medium, lot #100322GSH).

Treatment and Maintenance of Cultures—Four cultures were included in each treatment group. Using a calibrated positive displacement pipette, a 15 uL volume of the test Rhamnolipid composition was applied to the center of each EFT-400 culture (once per day; every 24 hours throughout testing). A sterile glass spreader was used to distribute the topical material across the surface. Each culture was visually inspected to ensure even distribution. Following topical applications, the cultures were returned to the incubator at 37° C. with 5% CO₂ and ˜95% relative humidity for 24 hours.

Tissue Collections-For each tissue collection, the topical material was washed from the surface of the culture with sterile DPBS. Following the removal of the topical material, each culture was placed into a tube containing RNAlater preservative solution. Tissues were incubated for 1-2 hours at room temperature and then transferred to a 4° C. refrigerator. Following a three day incubation in RNAlater solution at 4° C., RNA was isolated from each tissue as described below.

LDH Cytotoxicity Assay—Culture medium from each tissue sample was diluted 1:10 with sterile Phosphate Buffered Saline (PBS). A background control (diluted culture medium that was not used for cell culture), a “Low Control” (diluted treatment medium collected from the Untreated culture wells), and a “High Control” (diluted culture medium collected from the 1% Triton X-100 treated culture wells) were included in the assay. Each diluted sample was added to an optically clear, flat-bottom 96-well plate in duplicate.

The LDH reaction mixture (Takara Bio) was prepared and added to each aliquot of diluted medium (1:1). The reaction plate was incubated for ˜20 minutes at room temperature, protected from light. Stopping solution (1.0N HCl) was then added to each well and absorbance was measured at 492 nm with a reference filter at 620 nm. Each sample absorbance value was calculated as the mean OD492-OD620 value for the duplicate reaction wells, with the blank absorbance value subtracted. The % Cytotoxicity was then calculated relative to the Untreated (negative control, set to 0% cytotoxicity) and the Triton X-100 treated (positive control, set to 100% cytotoxicity) absorbance values, according to kit instructions: % Cytotoxicity=[(Test Media Value−Low Control)/(High Control-Low Control)]×100.

RNA Isolation—RNA was isolated from each tissue using the Maxwell RSC SimplyRNA Kit (Promega) following the manufacturer's instructions. The RNA samples were vacuum-concentrated until the concentration was at or above 115 ng/uL. RNA concentration and purity were determined using a Synergy H1 Microplate Reader (BioTek).

cDNA synthesis—cDNA was generated using a High Capacity cDNA Synthesis Kit (Applied Biosystems) according to the manufacturer's instructions. cDNA was generated from 1150 ng RNA per sample.

qPCR Processing—qPCR reactions were run using validated Taqman® gene expression assays in a 384-well plate format. Plates were run in a Life Technologies QuantStudio 12K Flex instrument. Each gene was assayed in duplicate.

Data Analysis—qPCR data quality and statistical analysis was assessed and performed on the raw data files using ThermoFisher Connect Software (Life Technologies). Technical replicates with high standard deviation were manually removed. Statistical analysis was performed using the relative quantitation (RQ) method. In the first step of an RQ analysis, the Cq value of the target gene is normalized to the Cq value of an endogenous control gene to generate the delta Cq (dCq). dCq values are calculated in order to normalize for variability between the samples that may occur during the experimental procedures.

Unpaired t-tests were carried out using Thermo Fisher Connect software. The statistical comparison generated delta delta Cq [dd Cq] values (the mean dCq of the treated group-the mean Cq of the control group). The statistical software converts the ddCq values into log and linear RQ values for export [RQ=2−ddcq]. The linear RQ values were converted to linear fold-change values to simplify data interpretation. Linear fold-change data was calculated from exported linear RQ values using Microsoft Excel: for RQ values≥1.0, Linear fold-change value=RQ value; and for RQ values<1.0, Linear fold-change value=−1/RQ value.

RNA Quality—Sample concentration and purity were determined using a Synergy H1 Microplate Reader. 260/280 readings indicate sample purity with ideal measurements that range from 1.8-2.1. All samples yielded high quality RNA.

Endogenous Control Gene Selection—An endogenous control gene that is consistently expressed in all of the samples of a comparison was selected. Two candidate control genes (hypoxanthine phosphoribosyltransferase 1 (HPRT1) and pptidylprolyl isomerase A (PPIA)) were tested. The most consistent endogenous control gene was chosen based on the stability score and range scores calculated using Thermo Fisher Data Connect RQ software. PPIA had the lowest range of Cq values and was selected as the endogeneous control gene for both the Day 3 and Day 5 testing below. Statistics (unpaired t-tests) were carried out for each comparison using dCq values normalized to the endogenous control gene.

qPCR Data Quality and Statistical Data Analysis—qPCR data quality is assessed using a combination of factors, including visual analysis of the shape of the qPCR curve and the Cq value. Cq values are an indication of the total amount of transcript present in the sample and can impact the quality of the qPCR data. qPCR amplification takes place over a total of 40 cycles, and typically occurs before cycle 30 in the OpenArray format (ThermoFisher Scientific). The relative amount of the gene transcript level is associated with the Cq value of the PCR reaction. Cq values typically correspond with the following: Cq values less than 30 are associated with high transcript levels and robust, high quality PCR data; and Cq values greater than 30 are associated with lower level transcripts, and less robust qPCR data.

qPCR Data Analysis—Relative Quantification (RQ) Data Analysis—In the first step of an RQ analysis, the Cq value of the endogenous control gene is subtracted from the Cq value of the target gene to generate the delta Cq (dCq). dCq values are calculated in order to normalize/control for variability between the samples that may occur during the experimental procedures.

For illustrative purposes, assume an example wherein: the mean Cq of endogenous control gene for Control as well as Treatment group samples=18 (no obvious separation between the Control group and the Treatment group); and the mean Cq of the target gene for the Control group samples=26 and for the Treatment group samples=17.

From the group Cq values, the group delta Cq values (dCq) are calculated for each target gene: the mean dCq for the Control Group sample: 26−18=8; and The mean dCq for the Treatment Group sample: 17−18=−1 (nine cycles earlier than the Control group). Amplification shifted to the left in the Treatment group (as in the example above) indicates higher levels of gene expression compared to the Control group. Amplification shifted to the right in the Treatment group would have indicated lower levels of gene expression compared to the Control group.

### Cytotoxicity (LDH Activity) Assay

Cytotoxicity was assessed with a lactate dehydrogenase (LDH) assay using media collected from the wells of the tissues at 1, 3 and 5 days. Increased LDH activity is an indicator of damaged or dead cells. Statistical data analysis (unpaired t-test, p□0.05) was performed to compare all of the groups to the Untreated Control Group. Tissues treated with Triton X-100 served as the positive control. For the LDH (+) control cultures (N=1), 100 uL of 1% Triton X-100 was applied to the surface of each culture; four Untreated cultures served as the LDH (−) control cultures. The Grubb's test was used to determine if there were outliers in this data set. None of the treatment groups had any meaningful impact on cytotoxicity at any of the time points (1 day, 3 days, or 5 days, after application of test material).

### Example 1

This study determined how certain rhamnolipid compositions impacted the gene expression profile of a number of genes, in skin responding to a wound. A 3D in vitro skin model containing epidermal keratinocytes and dermal fibroblasts was used. The model, MatTek Life Sciences (MatTek EFT-400), consists of normal, human-derived epidermal keratinocytes (NHEK) and normal, human-derived dermal Fibroblasts (NHFB) which have been cultured to form a multilayered, highly differentiated model of the human dermis and epidermis.

EFT-400 tissues (MatTek lot #34294, kits FF, EE, and Z-HCF) were equilibrated overnight at 37° C. with 5% CO2 and ˜95% relative humidity. The following day, equilibration medium was removed from each well and replaced with 2.5 ml fresh maintenance medium (MatTek EFT medium, lot #100322GSH). Tissues were wounded with a sterile 3 mm dermal biopsy punch, and gene expression was then assessed for 15 target genes, including: actin alpha 2 (ACTA2); ADAM metallopeptidase domain 17 (ADAM17); bone morphogenetic protein 6 (BMP6); CD14 molecule (CD14); claudin 1 (CLDN1); heparin binding EGF like growth factor (HBEGF); hepatocyte growth factor (HGF); hypoxia inducible factor 1 subunit alpha (HIF1A); intercellular adhesion molecule 1 (ICAM1); integrin subunit beta 1 (ITGB1); occludin (OCLN); plakophilin 1 (PKP1); peroxisome proliferator activated receptor delta (PPARD); transforming growth factor beta 1 (TGFB1); toll like receptor 3 (TLR3) and 2 endogenous control genes.

Four cultures were included in each treatment group. Using a calibrated positive displacement pipette, a 15 uL volume of each rhamnolipid composition was applied to the center of each EFT-400 culture at T=0 hours, and every 24 hours (day) thereafter. A sterile glass spreader was used to distribute the topical material across the surface. Each culture was visually inspected to ensure even distribution. Following topical applications, the cultures were returned to an incubator at 37° C. with 5% CO₂ and ˜95% relative humidity for 24 hours.

For each tissue collection, the topical material was washed from the surface of the culture with sterile DPBS. Following the removal of the topical material, each culture was placed into a tube containing RNAlater preservative solution (ThermoFisher Scientific). Tissues were incubated for 1-2 hours at room temperature and then transferred to a 4° C. refrigerator.

Following a three day incubation in RNAlater at 4° C., RNA was isolated from each tissue using the Maxwell RSC SimplyRNA Kit (Promega) following the manufacturer's instructions. The RNA samples were vacuum-concentrated until the concentration was at or above 115 ng/uL. RNA concentration and purity were determined using a Synergy Hl Microplate Reader.

cDNA was generated, from 1150 ng RNA per sample, using a High Capacity cDNA Synthesis Kit according to the manufacturer's instructions (Applied Biosystems). Quantitative PCR (qPCR) reactions were run using validated Taqman® gene expression assays in a 384-well plate format. Plates were run in a Life Technologies QuantStudio 12K Flex instrument. Each gene was assayed in duplicate.

qPCR data quality and statistical analysis was assessed and performed on the raw data files using ThermoFisher Connect Software (Life Technologies). Technical replicates with high standard deviation were manually removed. Statistical analysis was performed using the relative quantitation (RQ) method. In the first step of an RQ analysis, the Cq value of the target gene is normalized to the Cq value of an endogenous control gene to generate the delta Cq (dCq). dCq values are calculated in order to normalize for variability between the samples that may occur during the experimental procedures.

Gene expression was assessed following either a 3-day (Table 1) or 5-day (Table 2) exposure to the following rhamnolipid test material, prepared as described below: SEP-RM V2 (SEP-RM rhamnolipid composition described above, with added bleaching step (occurring after acidulation, and before the solvent extraction)); Mono-rhamnolipid R95Md (Sigma-Aldrich); Di-rhamnolipid R95Dd (Sigma-Aldrich); artificially formulated 50/50 Mono-rhamnolipid/Di-rhamnolipid mix (0.2% Mono-rhamnolipid (R95Md (Sigma-Aldrich))+0.2% Di-rhamnolipid R95Dd (Sigma-Aldrich) in Saline); and a commercially available comparator rhamnolipid formulation. The test rhamnolipids were stored at room temperature and protected from light until use. The working stock of each rhamnolipid used in this Example test material was prepared using 0.9% saline (Moltox Molecular Toxicology, Inc., lot #56205).

Statistically significant (unpaired t-test, p.50.05, N=4) changes in gene expression, for the rhamnolipid groups specified above, versus Saline at three days are presented below in Table 1. Included are linear fold change values (FA), and percent change values (% Δ).

TABLE 1
Statistically Significant Changes in Gene Expression
Relative to Negative Control (Saline) at Day 3

| | vs 0.9% Saline | | | | | | | | | |
|---|---|---|---|---|---|---|---|---|---|---|
| | | | | | | | 50/50 Mono-Rh | | | |
| | | | Mono-Rh | | Di-Rh | | (R95Md)/Di-Rh | | Commercial | |
| Target | SEP-RM V2 | | (R95Md) | | (R95Dd) | | (R95Dd) | | Comparator | |
| Name | FΔ | % Δ | FΔ | % Δ | FΔ | % Δ | FΔ | % Δ | FΔ | % Δ |
| ACTA2 | −2.16 | −54 | −2.28 | −56 | −1.69 | −41 | −1.55 | −36 | −1.28 | −0.221 |
| ADAM17 | 1.13 | 13 | 1.11 | 11 | −1.00 | 0 | −1.06 | −6 | −1.02 | −0.016 |
| BMP6 | 4.31 | 331 | 6.74 | 574 | 2.77 | 177 | 1.89 | 89 | −1.52 | −0.344 |
| CD14 | −1.18 | −16 | −1.30 | −23 | 1.23 | 23 | −1.01 | −1 | 1.55 | 0.545 |
| CLDN1 | −1.34 | −26 | −1.52 | −34 | −1.28 | −22 | −1.22 | −18 | 1.00 | 0.004 |
| HBEGF | 3.79 | 279 | 3.38 | 238 | 2.44 | 144 | 2.43 | 143 | 2.04 | 1.042 |
| HGF | 2.28 | 128 | 2.99 | 199 | 1.77 | 77 | 1.20 | 20 | −1.15 | −0.134 |
| HIF1A | 1.20 | 20 | 1.10 | 10 | 1.10 | 10 | 1.02 | 2 | 1.05 | 0.05 |
| ICAM1 | 1.77 | 77 | 1.97 | 97 | 1.41 | 41 | 1.14 | 14 | 1.03 | 0.026 |
| ITGB1 | 1.40 | 40 | 1.49 | 49 | 1.13 | 13 | −1.07 | −6 | −1.07 | −0.066 |
| OCLN | 2.16 | 116 | 1.88 | 88 | 1.50 | 50 | 1.48 | 48 | 1.32 | 0.316 |
| PKP1 | 1.10 | 10 | −1.13 | −11 | 1.12 | 12 | 1.28 | 28 | 1.19 | 0.192 |
| PPARD | 1.99 | 99 | 1.50 | 50 | 1.46 | 46 | 1.23 | 23 | 1.15 | 0.153 |
| TGFB1 | −1.02 | −2 | −1.08 | −8 | −1.09 | −9 | −1.11 | −10 | −1.23 | −0.188 |
| TLR3 | −3.42 | −71 | −6.58 | −85 | −1.58 | −37 | −1.57 | −36 | 1.23 | 0.231 |

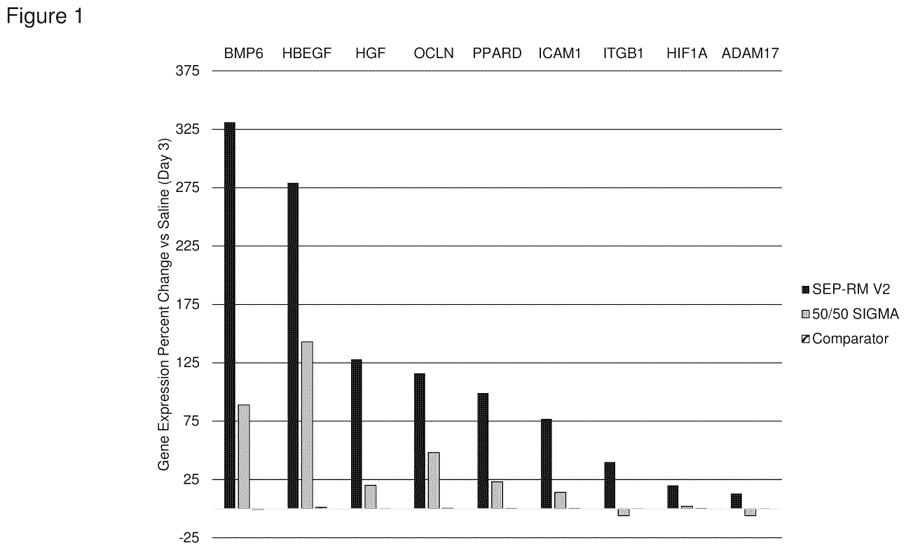

As set forth in Table 1 above, and as illustrated in FIGS. 1-3, a 3-day exposure to SEP-RM Rhamnolipid resulted in a significantly different gene expression profile, compared to the same 3-day exposure to an artificially generated 50/50 Mono-rhamnolipid/Di-rhamnolipid formulation (0.2% Mono-rhamnolipid (R95Md (Sigma-Aldrich))+0.2% Di-rhamnolipid R95Dd (Sigma-Aldrich) in Saline), or to a commercially available comparator rhamnolipid formulation.

For example, as illustrated in FIG. 1, BMP6, HBEGF, HGF, OCLN, PPARD, ICAM1, ITGB1, HIF1A, AND ADAM17 gene expression is activated, relative to the negative control 0.9% saline, after a 3-day exposure to SEP-RM Rhamnolipid V2, and activated to a greater extent compared to an artificially generated 50/50 Mono-rhamnolipid/Di-rhamnolipid formulation, and to a commercially available comparator rhamnolipid formulation.

In one aspect of the present disclosure, there is provided a rhamnolipid composition for use in a method of treating a wound, wherein application of the composition increases BMP6 gene expression, within 3 days after application of the composition, wherein the increase in BMP6 gene expression is from 2 to 6 fold, from 3 to 5 fold, or about 4 fold, relative to a saline negative control. Likewise, the increase in BMP6 gene expression has a percent change of from 200% to 400%, from 300% to 400%, from 300% to 350%, or from 350% to 400%, relative to a saline negative control.

In another aspect of the present disclosure, there is provided a rhamnolipid composition for use in a method of treating a wound, wherein application of the composition increases HBEGF gene expression, within 3 days after application of the composition, wherein the increase in HBEGF gene expression is from 2 to 6 fold, from 3 to 5 fold, from 3 to 4 fold, from 3.5 to 4 fold, or about 4 fold, relative to a saline negative control. Likewise, the increase in HBEGF gene expression has a percent change of from 200% to 400%, from 200% to 300%, or from 250% to 300%, relative to a saline negative control.

In another aspect of the present disclosure, there is provided a rhamnolipid composition for use in a method of treating a wound, wherein application of the composition increases HGF gene expression, within 3 days after application of the composition, wherein the increase in HGF gene expression is from 1 to 4 fold, from 1 to 3 fold, from 2 to 3 fold, from 2 to 2.5 fold, or about 2 fold, relative to a saline negative control. Likewise, the increase in HGF gene expression has a percent change of from 100% to 200%, from 100% to 150%, from 100% to 125%, or about 125%, relative to a saline negative control.

In a still further aspect of the present disclosure, there is provided a rhamnolipid composition for use in a method of treating a wound, wherein application of the composition increases OCLN gene expression, within 3 days after application of the composition, wherein the increase in OCLN gene expression is from 1 to 4 fold, from 1 to 3 fold, from 2 to 3 fold, from 2 to 2.5 fold, or about 2 fold, relative to a saline negative control. Likewise, the increase in OCLN gene expression has a percent change of from 100% to 150%, from 100% to 125%, or about 125%, relative to a saline negative control.

In a still further aspect of the present disclosure, there is provided a rhamnolipid composition for use in a method of treating a wound, wherein application of the composition increases PPARD gene expression, within 3 days after application of the composition, wherein the increase in PPARD gene expression is from 1 to 4 fold, from 1 to 3 fold, from 1 to 2 fold, or about 2 fold, relative to a saline negative control. Likewise, the increase in PPARD gene expression has a percent change of from 50% to 150%, 75% to 125%, from 50% to 100%, from 75% to 100%, or about 100%, relative to a saline negative control.

In a still further aspect of the present disclosure, there is provided a rhamnolipid composition for use in a method of treating a wound, wherein application of the composition increases ICAM1 gene expression, within 3 days after application of the composition, wherein the increase in ICAM1 gene expression is from 1 to 4 fold, from 1 to 3 fold, from 1 to 2 fold, from 1 to 1.75 fold, or about 1.75 fold, relative to a saline negative control. Likewise, the increase in ICAM1 gene expression has a percent change of from 50% to 100%, 50% to 75%, or about 75%, relative to a saline negative control.

In a still further aspect of the present disclosure, there is provided a rhamnolipid composition for use in a method of treating a wound, wherein application of the composition increases ITGB1 gene expression, within 3 days after application of the composition, wherein the increase in ITGB1 gene expression is from 1 to 3 fold, from 1 to 2 fold, from 1 to 1.50 fold, or about 1.50 fold, relative to a saline negative control. Likewise, the increase in ITGB1 gene expression has a percent change of from 20% to 60%, 30% to 50%, or about 40%, relative to a saline negative control.

In a still further aspect of the present disclosure, there is provided a rhamnolipid composition for use in a method of treating a wound, wherein application of the composition increases HIF1A gene expression, within 3 days after application of the composition, wherein the increase in HIF1A gene expression is from 1 to 2 fold, from 1 to 1.50 fold, from 1 to 1.25 fold or about 1.25 fold, relative to a saline negative control. Likewise, the increase in HIF1A gene expression has a percent change of from 10% to 30%, 10% to 20%, or about 20%, relative to a saline negative control.

In a still further aspect of the present disclosure, there is provided a rhamnolipid composition for use in a method of treating a wound, wherein application of the composition increases ADAM17 gene expression, within 3 days after application of the composition, wherein the increase in ADAM17 gene expression is from 1 to 2 fold, from 1 to 1.50 fold, from 0.75 to 1.25 fold, or about 1.15 fold, relative to a saline negative control. Likewise, the increase in ADAM17 gene expression has a percent change of from 10% to 30%, 10% to 20%, 10% to 15%, or about 15%, relative to a saline negative control.

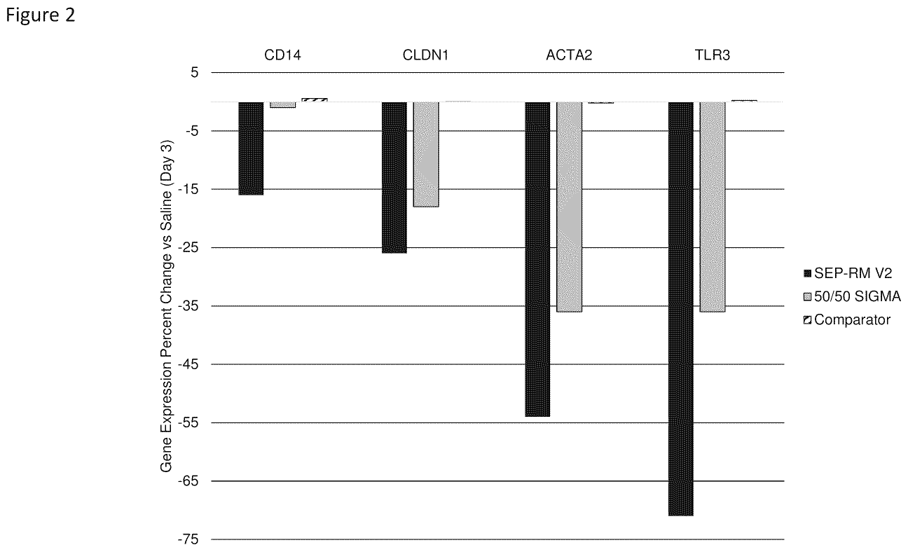

As illustrated in FIG. 2, CD14, CLDN1, ACTA2, and TLR3 gene expression is suppressed, relative to the negative control 0.9% saline, after a 3-day exposure to SEP-RM Rhamnolipid V2, and suppressed to a greater extent compared to an artificially generated 50/50 Mono-rhamnolipid/Di-rhamnolipid formulation, and to a commercially available comparator rhamnolipid formulation.

In one aspect of the present disclosure, there is provided a rhamnolipid composition for use in a method of treating a wound, wherein application of the composition decreases CD14 gene expression, within 3 days after application of the composition, wherein the decrease in CD14 gene expression is from 0.5 to 2 fold, from 0.5 to 1.5 fold, from 1 to 1.5 fold, or about 1.25 fold, relative to a saline negative control. Likewise, the decrease in CD14 gene expression has a percent change of from −10% to −20%, −10% to −15%, or about −15%, relative to a saline negative control.

In one aspect of the present disclosure, there is provided a rhamnolipid composition for use in a method of treating a wound, wherein application of the composition decreases CLDN1 gene expression, within 3 days after application of the composition, wherein the decrease in CLDN1 gene expression is from 1 to 2 fold, from 1 to 1.5 fold, or about 1.25 fold, relative to a saline negative control. Likewise, the decrease in CLDN1 gene expression has a percent change of from −10% to −40%, −10% to −30%, −15% to −25%, or about −25%, relative to a saline negative control.

In one aspect of the present disclosure, there is provided a rhamnolipid composition for use in a method of treating a wound, wherein application of the composition decreases ACTA2 gene expression, within 3 days after application of the composition, wherein the decrease in ACTA2 gene expression is from 1 to 3 fold, from 1 to 2.5 fold, from 1 to 2 fold, or about 2 fold, relative to a saline negative control. Likewise, the decrease in ACTA2 gene expression has a percent change of from −25% to −100%, −25% to −75%, −25% to −50%, or about −50%, relative to a saline negative control.

In one aspect of the present disclosure, there is provided a rhamnolipid composition for use in a method of treating a wound, wherein application of the composition decreases TLR3 gene expression, within 3 days after application of the composition, wherein the decrease in TLR3 gene expression is from 1 to 5 fold, from 2 to 4 fold, from 3 to 4 fold, from 3 to 3.5 fold, or about 3.5 fold, relative to a saline negative control. Likewise, the decrease in TLR3 gene expression has a percent change of from −25% to −100%, −25% to −75%, −50% to −75%, or about −75%, relative to a saline negative control.

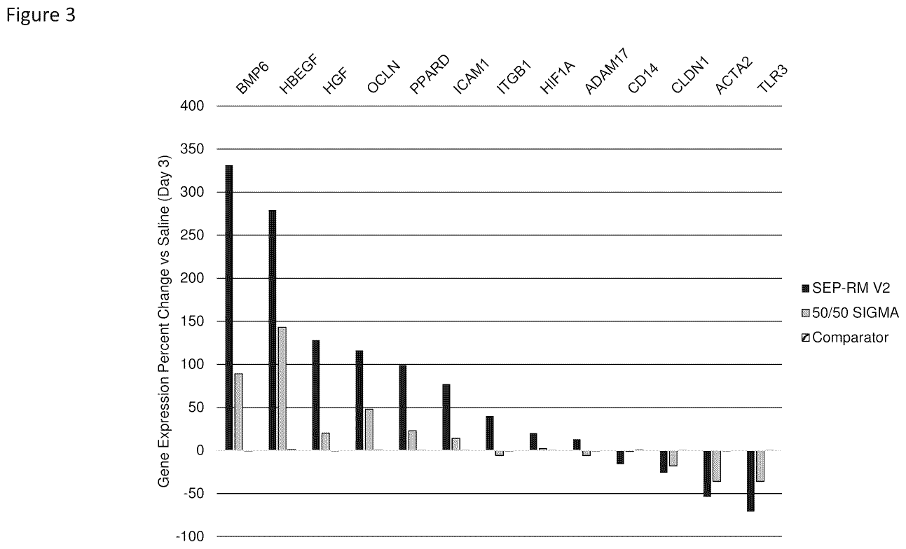

FIG. 3 illustrates the overall gene expression profile (including both gene activation and suppression after a 3-day exposure to SEP-RM Rhamnolipid V2, an artificially generated 50/50 Mono-rhamnolipid/Di-rhamnolipid formulation (0.2% Mono-rhamnolipid (R95Md (Sigma-Aldrich))+0.2% Di-rhamnolipid R95Dd (Sigma-Aldrich) in Saline), and to a commercially available comparator rhamnolipid formulation.

### Example 2

Example 1 was repeated except that changes in gene expression, for the rhamnolipid groups specified above, were evaluated at 5 days.

Statistically significant (unpaired t-test, p.50.05, N=4) changes in gene expression, for the rhamnolipid groups specified above, versus Saline at five days are presented below in Table 2. Included are linear fold change values (FA), and percent change values (% A).

TABLE 2
Statistically Significant Changes in Gene Expression
Relative to Negative Control (Saline) at Day 5

| | vs 0.9% Saline | | | | | | | | | |
|---|---|---|---|---|---|---|---|---|---|---|
| | | | | | | | 50/50 Mono-Rh | | | |
| | | | Mono-Rh | | Di-Rh | | (R95Md)/Di-Rh | | Commercial | |
| Target | SEP-RM V2 | | (R95Md) | | (R95Dd) | | (R95Dd) | | Comparator | |
| Name | FΔ | % Δ | FΔ | % Δ | FΔ | % Δ | FΔ | % Δ | FΔ | % Δ |
| ACTA2 | 1.3 | 30 | 1.36 | 36 | 1.54 | 54 | 2.54 | 154 | 2.00 | 100 |
| ADAM17 | 1.01 | 0 | 1.09 | 9 | −1.09 | −8 | 1.75 | 75 | 1.74 | 74 |
| BMP6 | −1.03 | −3 | −1.22 | −18 | −1.09 | −8 | 1.75 | 75 | 1.08 | 8 |
| CD14 | 1.35 | 35 | 1.67 | 67 | 1.72 | 72 | 2.30 | 130 | 1.88 | 88 |
| CLDN1 | −1.57 | −36 | −1.53 | −35 | −5.10 | −80 | 1.53 | 53 | 2.20 | 120 |
| HBEGF | −1.13 | −12 | −1.20 | −17 | −4.05 | −75 | 1.63 | 63 | 2.75 | 175 |
| HGF | 2.08 | 108 | 1.81 | 81 | 2.90 | 190 | 2.87 | 187 | 1.61 | 61 |
| HIF1A | 1.18 | 18 | 1.13 | 13 | 1.30 | 30 | 1.90 | 90 | 1.83 | 83 |
| ICAM1 | 1.09 | 9 | −1.04 | −4 | −1.11 | −10 | 1.23 | 23 | 1.80 | 80 |
| ITGB1 | 1.81 | 81 | 1.75 | 75 | 1.97 | 97 | 2.80 | 180 | 1.90 | 90 |
| OCLN | −1.33 | −25 | −1.40 | −29 | −3.57 | −72 | −1.09 | −8 | −1.43 | −30 |
| PKP1 | −1.38 | −28 | −1.28 | −22 | −6.41 | −84 | −1.07 | −7 | 1.41 | 41 |
| PPARD | −2.73 | −63 | −1.91 | −48 | −2.35 | −58 | −2.43 | −59 | −1.19 | −16 |
| TGFB1 | 1.03 | 3 | 1.43 | 43 | 1.09 | 9 | 1.31 | 31 | 1.22 | 22 |
| TLR3 | −1.69 | −41 | 1.64 | 64 | −1.37 | −27 | −2.82 | −65 | 1.13 | 13 |

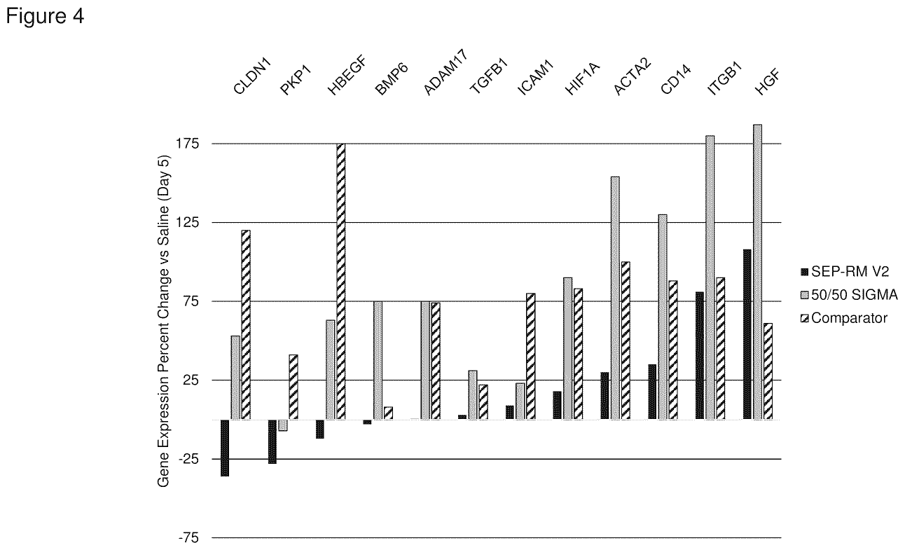

As set forth in Table 2 above, and as illustrated in FIG. 4, a 5-day exposure to SEP-RM V2 Rhamnolipid resulted in a significantly different gene expression profile, compared to exposure to an artificially generated 50/50 Mono-rhamnolipid/Di-rhamnolipid formulation, and to a commercially available comparator rhamnolipid formulation. In contrast to gene expression after a 3-day exposure to Rhamnolipid SEP-RM V2 (Tables 1, and FIGS. 1-3), gene expression after a 5-day exposure to Rhamnolipid SEP-RM V2 showed diminishing gene expression compared to exposure with the artificially generated 50/50 Mono-rhamnolipid/Di-rhamnolipid formulation (0.2% Mono-rhamnolipid (R95Md (Sigma-Aldrich))+0.2% Di-rhamnolipid R95Dd (Sigma-Aldrich) in Saline), and to the commercially available comparator rhamnolipid formulation.

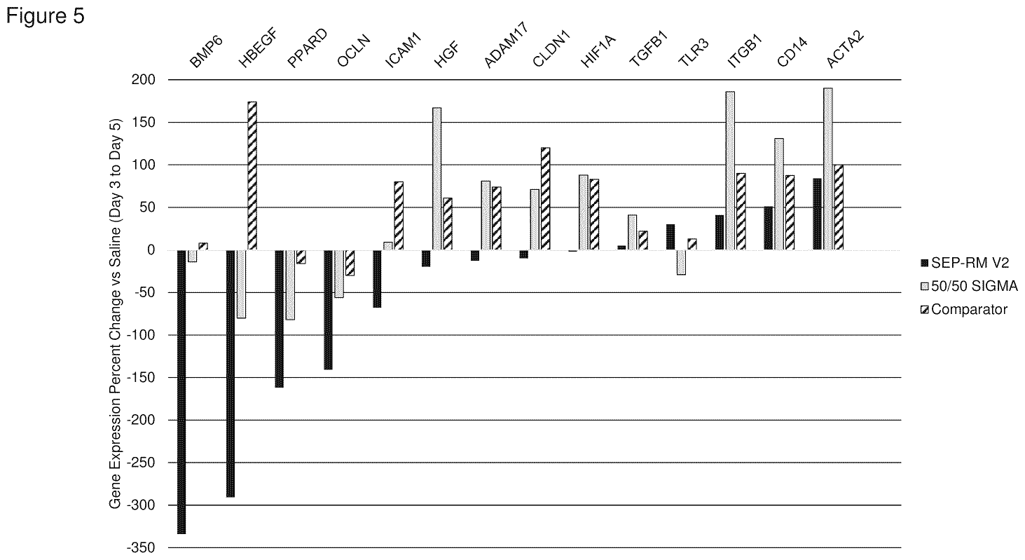

FIG. 5 is a graph showing the percent change in gene expression from 3 days to 5 days, relative to 0.9% saline, after exposure to Rhamnolipid SEP-RM V2 (with related comparison to 50/50 Mono-rhamnolipid/Di-rhamnolipid mix (0.2% Mono-rhamnolipid (R95Md (Sigma-Aldrich))+0.2% Di-rhamnolipid R95Dd (Sigma-Aldrich) in Saline), and a commercially available comparator rhamnolipid formulation). As illustrated in FIG. 5, the changes in gene expression from day 3 to day 5 is significantly different for each rhamnolipid composition. For example, as illustrated in FIG. 5, after exposure to Rhamnolipid SEP-RM V2 gene expression is decreasing, from day 3 to day 5, for genes BMP6, HBEGF, PPARD, OCLN, ICAM1, HGF, ADAM17, and CLDN1.

The present technology is now described in such full, clear and concise terms as to enable a person skilled in the art to which it pertains, to practice the same. It is to be understood that the foregoing describes preferred embodiments of the present technology and that modifications may be made therein without departing from the spirit or scope of the present technology as set forth in the appended claims. Further, the examples are provided to not be exhaustive but illustrative of several embodiments that fall within the scope of the claims.

1. A composition for use in a method of treating a wound, the composition comprising:
(a) a mixture of rhamnolipids, wherein the mixture of rhamnolipids comprises:
mono-rhamnolipids and di-rhamnolipids in a weight ratio of about 40:60 to about 60:40 mono-rhamnolipids: di-rhamnolipids;
an amount of C10-C10 mono-rhamnolipid of about 29% to about 40% by weight, based on the total weight of rhamnolipids present in the composition;
an amount of C10-C10 di-rhamnolipid of about 35% to about 50% by weight, based on the total weight of rhamnolipids present in the composition;
an amount of C8-C10 mono-rhamnolipid of about 2% to about 5% by weight, based on the total weight of rhamnolipids present in the composition;
an amount of C8-C10 di-rhamnolipid of about 2% to about 5% by weight, based on the total weight of rhamnolipids present in the composition;
an amount of C10-C12 mono-rhamnolipid of about 2% to about 6% by weight, based on the total weight of rhamnolipids present in the composition; and
an amount of C10-C12 di-rhamnolipid of about 8% to about 14% by weight, based on the total weight of rhamnolipids present in the composition;
(b) at least one acceptable carrier, and optionally one or more additives, in an amount to total 100% by weight of the composition.

2. The composition for use in a method of treating a wound of claim 1, wherein the mixture of rhamnolipids further comprises an amount of C10-C12:1 di-rhamnolipid of about 2% to about 5% by weight, based on the total weight of rhamnolipids present in the composition.

3. The composition for use in a method of treating a wound of claim 1, wherein the mixture of rhamnolipids further comprises an amount of C12-C12 di-rhamnolipid of about 0.2% to about 0.4% by weight, based on the total weight of rhamnolipids present in the composition.

4. The composition for use in a method of treating a wound of claim 1, wherein the mixture of rhamnolipids comprises an amount of total mono-rhamnolipid of about 40% to about 50% by weight, based on the total weight of rhamnolipids present in the composition.

5. The composition for use in a method of treating a wound of claim 1, wherein the mixture of rhamnolipids comprises an amount of total di-rhamnolipid of about 50% to about 60% by weight, based on the total weight of rhamnolipids present in the composition.

6. The composition for use in a method of treating a wound of claim 1, wherein the mixture of rhamnolipids is in an amount of about 0.1% to about 10%, based on the total weight of the composition.

7. The composition for use in a method of treating a wound of claim 1, wherein the wound being treated is an incision, laceration, abrasion, or burn.

8. The composition for use in a method of treating a wound of claim 1, wherein the at least one acceptable carrier is water alone or in combination with an alcohol or glycol.

9. The composition for use in a method of treating a wound of claim 8, wherein the alcohol is ethanol, isopropanol, or benzyl alcohol.

10. The composition for use in a method of treating a wound of claim 8, wherein the glycol is propylene glycol or polyethylene glycol.

11. The composition for use in a method of treating a wound of claim 1, wherein the method comprises topical administration of the composition.

12. A wound care composition, comprising:
(a) a mixture of rhamnolipids, wherein the mixture of rhamnolipids comprises:
mono-rhamnolipids and di-rhamnolipids in a weight ratio of about 40:60 to about 50:50 mono-rhamnolipids: di-rhamnolipids;
an amount of C10-C10 mono-rhamnolipid of about 29% to about 37% by weight, based on the total weight of rhamnolipids present in the composition;
an amount of C10-C10 di-rhamnolipid of about 35% to about 50% by weight, based on the total weight of rhamnolipids present in the composition;
an amount of C8-C10 mono-rhamnolipid of about 2% to about 5% by weight, based on the total weight of rhamnolipids present in the composition;
an amount of C8-C10 di-rhamnolipid of about 2% to about 5% by weight, based on the total weight of rhamnolipids present in the composition;
an amount of C10-C12 mono-rhamnolipid of about 2% to about 6% by weight, based on the total weight of rhamnolipids present in the composition; and
an amount of C10-C12 di-rhamnolipid of about 8% to about 14% by weight, based on the total weight of rhamnolipids present in the composition;
(b) at least one acceptable carrier, and optionally one or more additives, in an amount to total 100% by weight of the composition.

13. The wound care composition of claim 12, wherein the mixture of rhamnolipids further comprises an amount of C10-C12:1 di-rhamnolipid of about 2% to about 5% by weight, based on the total weight of rhamnolipids present in the composition.

14. The wound care composition of claim 12, wherein the mixture of rhamnolipids further comprises an amount of C12-C12 di-rhamnolipid of about 0.2% to about 0.4% by weight, based on the total weight of rhamnolipids present in the composition.

15. The wound care composition of claim 12, wherein the mixture of rhamnolipids comprises an amount of total mono-rhamnolipid of about 40% to about 48% by weight, based on the total weight of rhamnolipids present in the composition.

16. The wound care composition of claim 12, wherein the mixture of rhamnolipids comprises an amount of total di-rhamnolipid of about 52% to about 60% by weight, based on the total weight of rhamnolipids present in the composition.

17. The wound care composition of claim 12, wherein the mixture of rhamnolipids is in an amount of about 0.1% to about 10%, based on the total weight of the composition.

18. The wound care composition of claim 12, wherein the at least one acceptable carrier is water alone or in combination with an alcohol or glycol.

19. The wound care composition of claim 18, wherein the alcohol is ethanol, isopropanol, or benzyl alcohol.

20. The wound care composition of claim 18, wherein the glycol is propylene glycol or polyethylene glycol.
